(19) United States
(12) Patent Application Publication
(10) Pub. No.: US 2017/0014087 A1
(43) Pub. Date: Jan. 19, 2017

(54) DEVICE, SYSTEM AND METHOD FOR DETERMINING VITAL SIGNS OF A SUBJECT

(71) Applicant: KONINKLIJKE PHILIPS N.V., EINDHOVEN (NL)

(72) Inventors: WILLEM VERKRUIJSSE, VELDHOVEN (NL); ALEXANDER DUBIELCZYK, GAERTRINGEN (DE); ROLF NEUMANN, CALW (DE)

(21) Appl. No.: 15/300,869

(22) PCT Filed: Mar. 19, 2015
(86) PCT No.: PCT/EP2015/055820

§ 371 Date: Sep. 30, 2016

(30) Foreign Application Priority Data
Mar. 31, 2014 (EP) 14162646.5

(51) Int. Cl. A61B 5/00; A61B 5/1455; A61B 5/145; A61B 5/01; A61B 5/021; A61B 5/024; A61B 5/08

(52) U.S. Cl. CPC A61B 5/7278; A61B 5/02416; A61B 5/14551; A61B 5/4809; A61B 5/02405; A61B 5/0816; A61B 5/01; A61B 5/021; A61B 5/14532

(57) ABSTRACT

The present invention relates to a device, system and method for determining vital signs of a subject. To balance accuracy and reliability with unobtrusiveness, the device comprises an interface (30) for receiving input signals from a detection unit (18) that is configured for contactless detection of radiation reflected from a subject (14) in response to illumination by an illumination source (22), a processing unit (32) for deriving photoplethysmography, PPG, signals from the received input signals, an analysis unit (34) for deriving a desired vital sign from the PPG signals, and a control unit (36) for controlling one or more parameters of the illumination source (22), the detection unit (18) and/or the analysis unit (34) in dependence on the physical state of the subject, the quality of the input signals or the derived vital sign and/or the value of the vital sign.

### FIELD OF THE INVENTION

The present invention relates to a device, system and method for determining vital signs of a subject, such as a person or animal.

### BACKGROUND OF THE INVENTION

Vital signs of a person, for example the heart rate (HR), the respiration rate (RR) or the arterial blood oxygen saturation, serve as indicators of the current state of a person and as powerful predictors of serious medical events. For this reason, vital signs are extensively monitored in inpatient and outpatient care settings, at home or in further health, leisure and fitness settings.

One way of measuring vital signs is plethysmography. Plethysmography generally refers to the measurement of volume changes of an organ or a body part and in particular to the detection of volume changes due to a cardio-vascular pulse wave traveling through the body of a subject with every heart beat.

Photoplethysmography (PPG) is an optical measurement technique that evaluates a time-variant change of light reflectance or transmission of an area or volume of interest. PPG is based on the principle that blood absorbs light more than surrounding tissue, so variations in blood volume with every heart beat affect transmission or reflectance correspondingly. Besides information about the heart rate, a PPG waveform can comprise information attributable to further physiological phenomena such as the respiration. By evaluating the transmittance and/or reflectivity at different wavelengths (typically red and infrared), the blood oxygen saturation can be determined.

Conventional pulse oximeters (also called contact PPG device herein) for measuring the heart rate and the (arterial) blood oxygen saturation (also called SpO2) of a subject are attached to the skin of the subject, for instance to a fingertip, earlobe or forehead. Therefore, they are referred to as ‘contact’ PPG devices. A typical pulse oximeter comprises a red LED and an infrared LED as light sources and one photodiode for detecting light that has been transmitted through patient tissue. Commercially available pulse oximeters quickly switch between measurements at a red and an infrared wavelength and thereby measure the transmittance of the same area or volume of tissue at two different wavelengths. This is referred to as time-division-multiplexing. The transmittance over time at each wavelength gives the PPG waveforms for red and infrared wavelengths. Although contact PPG is regarded as a basically non-invasive technique, contact PPG measurement is often experienced as being unpleasant and obtrusive, since the pulse oximeter is directly attached to the subject and any cables limit the freedom to move and might hinder a workflow.

Fast and reliable detection and analysis of a pulse signal and oxygen saturation level (SPO2) is one of the most important activities in many healthcare applications, which becomes crucial if a patient is in a critical condition. In those situations, pulsatility of a heart beat signal is very weak, and therefore, the measurement is vulnerable to any sort of artifacts.

Modern photoplethysmography sensors do not always provide fast and reliable measurement in critical situations. For instance, contact finger pulse oximeters (based on transmissive PPG) are vulnerable to motion of a hand, and fails in case of centralization of a patient due to lower blood volumes on body peripherals. Contact forehead pulse oximeter sensors (using a reflective PPG measurement mode) are supposed to be more robust to a centralization effect. However, the accuracy, robustness and responsiveness of a forehead sensor depends heavily on correct positioning of a sensor on a forehead and proper pressure applied to a skin (too tight application of a sensor might reduce a local blood pulsatility, too loose application might lead to non-reliable measurements due to motion artifacts and/or venous pulsatility).

Recently, non-contact, remote PPG (rPPG) devices (also called camera rPPG device herein) for unobtrusive measurements have been introduced. Remote PPG utilizes light sources or, in general radiation sources, disposed remotely from the subject of interest. Similarly, also a detector, e.g., a camera or a photo detector, can be disposed remotely from the subject of interest. Therefore, remote photoplethysmographic systems and devices are considered unobtrusive and well suited for medical as well as non-medical everyday applications. However, remote PPG devices typically achieve a lower signal-to-noise ratio.

Verkruysse et al., “Remote plethysmographic imaging using ambient light”, Optics Express, 16(26), 22 December 2008, pp. 21434-21445 demonstrates that photoplethysmographic signals can be measured remotely using ambient light and a conventional consumer level video camera, using red, green and blue colour channels.

Wieringa, et al., “Contactless Multiple Wavelength Photoplethysmographic Imaging: A First Step Toward “SpO2 Camera” Technology,” Ann. Biomed. Eng. 33, 1034-1041 (2005), discloses a remote PPG system for contactless imaging of arterial oxygen saturation in tissue based upon the measurement of plethysmographic signals at different wavelengths. The system comprises a monochrome CMOS-camera and a light source with LEDs of three different wavelengths. The camera sequentially acquires three movies of the subject at the three different wavelengths. The pulse rate can be determined from a movie at a single wavelength, whereas at least two movies at different wavelengths are required for determining the oxygen saturation. The measurements are performed in a darkroom, using only one wavelength at a time.

Using PPG technology, vital signs can be measured, which are revealed by minute light absorption changes in the skin caused by the pulsating blood volume, i.e. by periodic color changes of the human skin induced by the blood volume pulse. As this signal is very small and hidden in much larger variations due to illumination changes and motion, there is a general interest in improving the fundamentally low signal-to-noise ratio (SNR). There still are demanding situations, with severe motion, challenging environmental illumination conditions, or high required accuracy of the application, where an improved robustness and accuracy of the vital sign measurement devices and methods is required, particularly for the more critical healthcare applications.

WO 2013/093690 A1 discloses an apparatus for use in monitoring the baroreceptor reflex in a user, the apparatus comprising a processor configured to process a signal output by a first sensor that is attached to or located proximate to a bed to determine when the user moves from a lying position on the bed to a sitting position, and to provide an indication of the baroreceptor reflex of the user by processing the signal to determine the change in the heart rate of the user that occurs as a result of moving from the lying position to the sitting position.

Several methods and devices for determining vital signs by use of photo-plethysmography are disclosed in US 2012/0197137 A1, US 2014/0031696 A1 and US 2011/0237912 A1.

### SUMMARY OF THE INVENTION

It an object of the present invention to provide an improved device, system and method for determining vital signs of a subject which provide an optimal balance between measurement accuracy and reliability on then one hand and low obtrusiveness for the subject on the other hand.

In a first aspect of the present invention a device for determining vital signs of a subject is presented, the device comprising:
an interface for receiving input signals from a detection unit that is configured for contactless detection of radiation reflected from a subject in response to illumination by an illumination source,
a processing unit for deriving photoplethysmography, PPG, signals from the received input signals,
an analysis unit for deriving a desired vital sign from the PPG signals,
a control unit for controlling one or more parameters of the illumination source, the detection unit and/or the analysis unit in dependence on the physical state of the subject, the quality of the input signals or the derived vital sign and/or the value of the vital sign,
   wherein the control unit is configured to control one or more parameters of the illumination source, the detection unit and/or the analysis unit such that the accuracy and reliability of one or more derived vital signs are increased in case of a decreasing health state of the subject and/or a decreasing quality of the input signals or the derived vital sign and that the unobtrusiveness of the illumination and detection of radiation is increased in case of a stable or increasing health state of the subject and/or a stable or increasing quality of the input signals or the derived vital sign.

In a further aspect of the present invention a corresponding method is presented.

In still a further aspect of the present invention a system for determining vital signs of a subject is presented, the system comprising:
an illumination source for illuminating the subject,
a detection unit for contactless detection of radiation reflected from a subject in response to said illumination to obtain input signals, and
a device as proposed herein for determining vital signs of the subject.

Preferred embodiments of the invention are defined in the dependent claims. It shall be understood that the claimed method and system have similar and/or identical preferred embodiments as the claimed device and as defined in the dependent claims.

The present invention is based on the idea to exploit information about one or more of the physical state of the subject, the quality of the input signals or the derived vital sign and/or the value of the vital sign. This information is used for controlling one or more parameters of the illumination source, the detection unit and/or the analysis unit. This control is performed such that a tradeoff is made between accuracy and reliability on then one hand and obtrusiveness for the subject on the other hand. In particular, in general the acquisition of the vital sign(s) shall be as unobtrusive as possible, but under certain conditions more accuracy and reliability may be required so that the parameters are controlled accordingly even if the acquisition will become less unobtrusive in this way.

Generally, the interaction of electromagnetic radiation, in particular light, with biological tissue is complex and includes the (optical) processes of (multiple) scattering, backscattering, absorption, transmission and (diffuse) reflection. The term “reflect” as used in the context of the present invention is not to be construed as limited to specular reflection but comprises the afore-mentioned types of interaction of electromagnetic radiation, in particular light, with tissue and any combinations thereof.

The term “vital sign” as used in the context of the present invention refers to a physiological parameter of a subject (i.e. a living being) and derivative parameters. In particular, the term “vital sign” comprises blood volume pulse-signal, heart rate (HR) (sometimes also called pulse rate), heart rate variability (pulse rate variability), pulsatility strength, perfusion, perfusion indicator, perfusion variability, Traube Hering Mayer waves, respiratory rate (RR), skin temperature, blood pressure, a concentration of a substance in blood and/or tissue, such as (arterial) blood oxygen saturation or glucose level. Furthermore, “vital sign” generally includes health indications obtained from the shape of the PPG signal (e.g. shape may say something about partial arterial blockage (e.g. shape obtained from PPG signals of the hand gets more sinusoidal when applying a blood-pressure cuff on the arm), or about the skin thickness (e.g. a PPG signal from the face is different than from the hand), or maybe even about the temperature, etc.).

The term “vital sign information” as used in the context of the present invention comprises the one or more measured vital signs as defined above. Furthermore, it comprises data referring to a physiological parameter, corresponding waveform traces or data referring to a physiological parameter of a time that can serve for subsequent analysis.

For obtaining a vital sign information signal of the subject the data signals of skin pixel areas within the skin area are evaluated. Here, a “skin pixel area” means an area comprising one skin pixel or a group of adjacent skin pixels, i.e. a data signal may be derived for a single pixel or a group of skin pixels.

According to the present invention said control unit is configured to control one or more parameters of the illumination source, the detection unit and/or the analysis unit such that the accuracy and reliability of one or more derived vital signs are increased in case of a decreasing health state of the subject and/or a decreasing quality of the input signals or the derived vital sign and that the unobtrusiveness of the illumination and detection of radiation is increased in case of a stable or increasing health state of the subject and/or a stable or increasing quality of the input signals or the derived vital sign.

Preferably, said control unit is configured to switch the illumination source, the detection unit and/or the device between at least a safety mode ensuring the acquisition of one or more accurate and reliable vital signs and a comfort mode ensuring an unobtrusive illumination and radiation detection. Conditions may be defined by a user or may be predefined, which are applied in the decision when to switch between the two different modes.

Various parameters may be controlled. In an embodiment said control unit is preferably configured to control one or more of the intensity, wavelength, direction and/or illumination angle of light emitted by illumination source. In another embodiment said control unit is configured to control acquisition rate, exposure time, focus, zoom or active sensing area of the detection unit. In still another embodiment said control unit is configured to control the analysis unit which vital signs to derive from the PPG signals.

Advantageously, said interface is configured to receive a sequence of image frames as input signal acquired by an imaging unit, in particular a camera, and said analysis unit is configured to obtain physical state information of the subject from said sequence of image frames.

Preferably, in an embodiment said analysis unit is configured to determine if the subject is in a physical state of moving or not moving, in particular in a physical state of sleeping or awake. This information may be exploited to use parameters that make the acquisition of vital signs less obtrusive if the subject is not moving or sleeping in order not to disturb the subject more than necessary, while it may be more obtrusive and thus more accurate/reliable if the subject is moving or awake.

In still another embodiment said control unit is configured to additionally use personal data of the subject, in particular age, gender, size, weight, health status, prior measurements of vital signs, health data, for controlling one or more parameters of the illumination source, the detection unit and/or the analysis unit.

### BRIEF DESCRIPTION OF THE DRAWINGS

These and other aspects of the invention will be apparent from and elucidated with reference to the embodiments described hereinafter. In the following drawings

FIG. 1 shows a schematic diagram of a system including a device according to the present invention,

FIG. 2 shows a more detailed embodiment of the proposed device, and

FIG. 3 shows various graphs for illustrating a preferred embodiment.

### DETAILED DESCRIPTION OF THE INVENTION

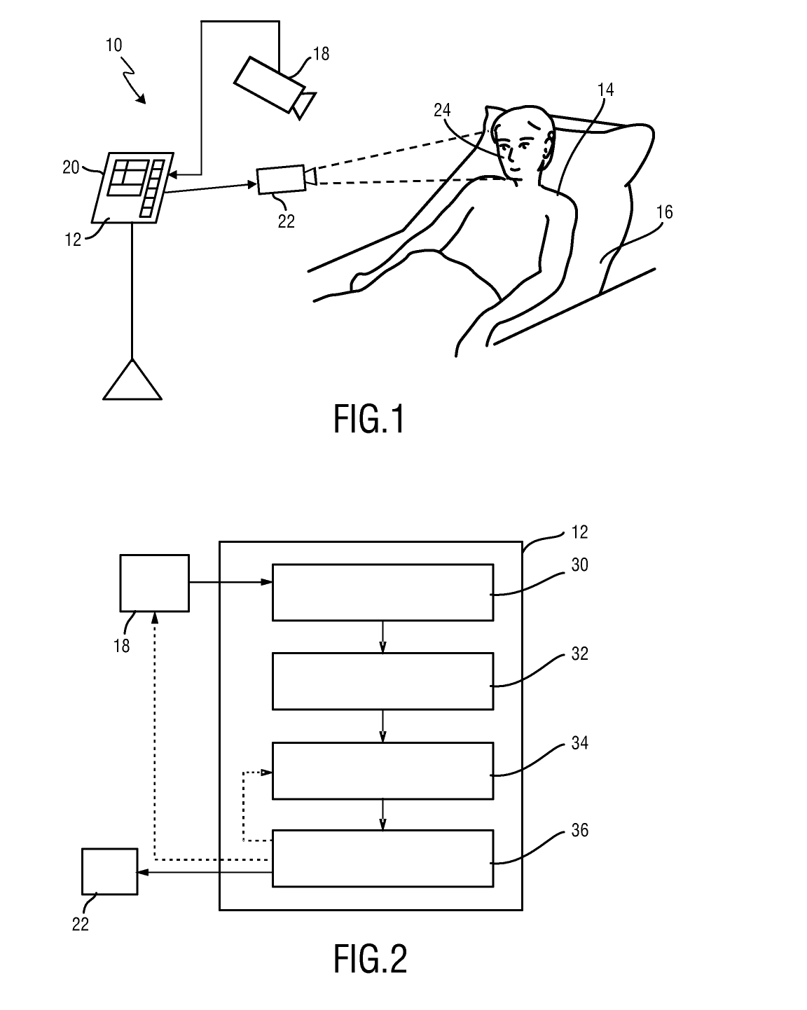

FIG. 1 shows a schematic diagram of a an embodiment of a system 10 including a device 12 for obtaining vital signs of a subject 14 according to the present invention. The subject 14, in this example a patient, lies in a bed 16, e.g. in a hospital or other healthcare facility, but may also be a neonate or premature infant, e.g. lying in an incubator, or person at home or in a different environment. Image frames of the subject 14 are captured by means of a camera 18 (also referred to as detection unit or as camera-based or remote PPG sensor) including a suitable photosensor. The camera 18 forwards the recorded image frames to processing means of the device 12, where the image frames will be processed as explained in more detail below. The device 12 preferably comprises an interface 20 for displaying the determined information and/or for providing medical personnel with an interface to change settings of the device 12 and/or other elements of the system 10. Such an interface 20 may comprise different displays, buttons, touchscreens, keyboards or other human machine interface means.

The system 10 further preferably comprises a light source 22 (also called illumination source), such as a lamp, for illuminating a region of interest 24, such as the skin of the patient's face (e.g. part of the cheek or forehead), with light, for instance in a predetermined wavelength range or ranges (e.g. in the red, green and/or infrared wavelength range(s)). The light reflected from said region of interest 24 in response to said illumination is detected by the camera 18. In another embodiment no dedicated light source is provided, but ambient light is used for illumination of the subject 14. From the reflected light only light in a desired wavelength range (e.g. green light) may be detected and/or evaluated.

The image frames captured by the camera 18 may particularly correspond to a video sequence captured by means of an analog or digital photosensor, e.g. in a (digital) camera. Such a camera 18 usually includes a photosensor, such as a CMOS or CCD sensor, which may also operate in a specific spectral range (visible, IR) or provide information for different spectral ranges. The camera 18 may provide an analog or digital signal. The image frames include a plurality of image pixels having associated pixel values. Particularly, the image frames include pixels representing light intensity values captured with different photosensitive elements of a photosensor. These photosensitive elements may be sensitive in a specific spectral range (i.e. representing a specific color). The image frames include at least some image pixels being representative of a skin portion of the subject. Thereby, an image pixel may correspond to one photosensitive element of a photo-detector and its (analog or digital) output or may be determined based on a combination (e.g. through binning) of a plurality of the photosensitive elements.

A system 10 as illustrated in FIG. 1 may, e.g., be located in a hospital, healthcare facility, elderly care facility, incubator or the like. Apart from the monitoring of patients, the present invention may also be applied in other fields such as neonate monitoring, general surveillance applications, security monitoring or so-called live style environments, such as fitness equipment, or the like. The uni- or bidirectional communication between the device 12, the camera 18 and the light source 22 may work via a wireless or wired communication interface, whereby it is to be noted that the light source 22 may also be configured to operate stand-alone and without communication with the device 12. Further, the device 12 and/or the light source 22 may also be incorporated into the camera 18.

FIG. 2 shows a more detailed schematic illustration of an embodiment of the device 12 according to the present invention. The device 12 comprises an interface 30 for receiving input signals from the detection unit, i.e. the camera 18, that is generally configured for contactless detection of radiation reflected from a subject 14 in response to illumination by the illumination source 22. The received input signals are processed by a processing unit 32 for deriving photoplethysmography (PPG) signals. The way to obtain PPG signals from detected light, e.g. from images of a region of interest, is generally known in the art, e.g. from the above cited documents, and will not be explained in more detail here. Still further, an analysis unit 34 is provided for deriving a desired vital sign from the PPG signals. Finally, the device 12 comprises a control unit 36 for controlling one or more parameters of the illumination source 22, the detection unit 18 and/or the analysis unit 34 in dependence on the physical state of the subject, the quality of the input signals or the derived vital sign and/or the value of the vital sign.

Novel camera-based methods enable remote, non-contact monitoring of vital signs such as pulse rate and arterial blood oxygenation (SpO2). To be able to measure SpO2 it is necessary to measure the normalized pulsatility at two wavelengths, typically red and near-infrared (NIR) (e.g. 660 nm and 800 nm, respectively). The contrast for changes in SpO2 (and thus accuracy/sensitivity) is highest when the red wavelength is chosen close to 660 nm. Unfortunately, light of this wavelength is quite visible for the human eye. Light of a higher wavelength, closer to IR (e.g. 750 nm) is much (about two orders of magnitude) less visible to the human eye but also provides a less beneficial contrast (sensitivity) for SpO2 changes. However, at this wavelength it may still be possible to perform accurate SpO2 measurements, but the measurement of the pulsatility amplitude should be done more precisely.

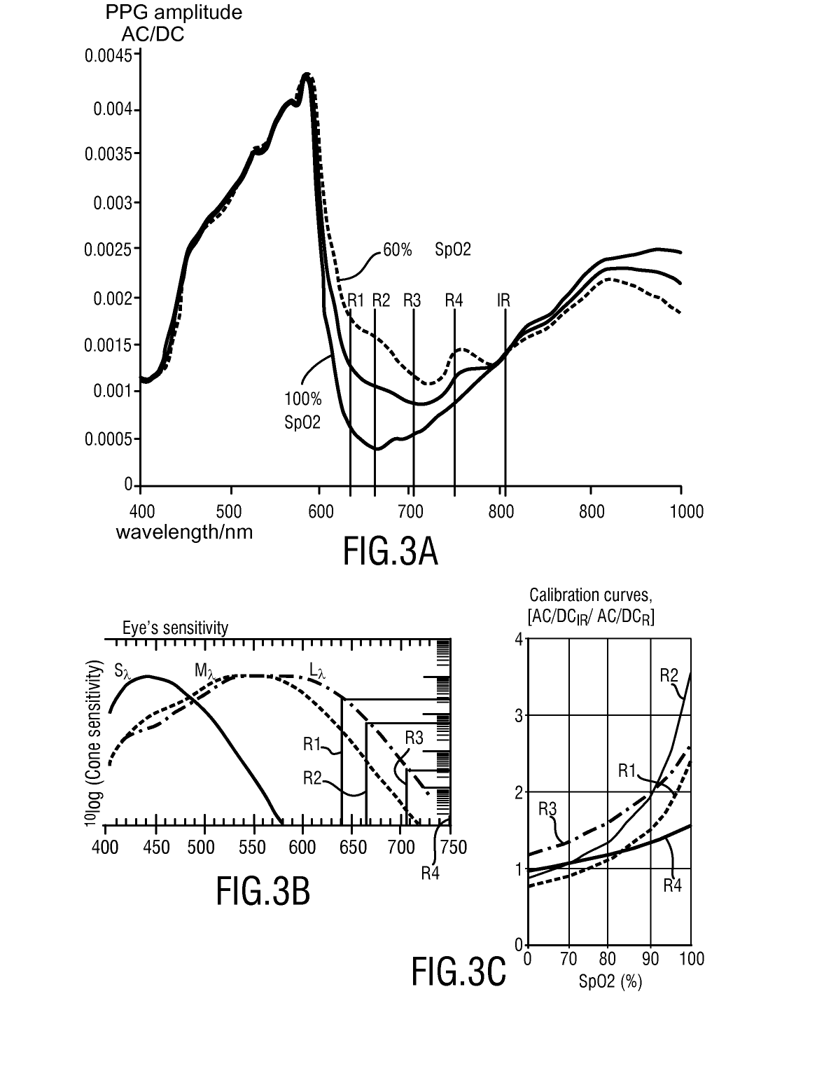

FIG. 3 shows various graphs illustrating a preferred embodiment of the proposed device. FIG. 3A shows PPG amplitude spectra for SpO2 levels of 100% (oxygenated arterial blood) down to 60% (low oxygenated arterial blood). To illustrate the relative contrast for SpO2 of the ratio of ratio's four different red wavelengths R1-R4 have been selected, while keeping the IR wavelength constant at 800 nm. FIG. 3B shows the sensitivity curves for the human eye illustrating the relative obtrusiveness of the wavelength choices (R4 at 750 nm is much less (more than 2 orders of magnitude) visible than R2 at 660 nm). In FIG. 3C the choice of R2 (660 nm) shows a high contrast for changes in SpO2 when compared to other choices. R4, for example, has a much smaller slope but the smaller visibility of this light is beneficial for sleep conditions.

The illumination of red light in camera based oximetry can thus be obtrusive and disturb optimal sleeping conditions. This problem is mitigated in an embodiment of the present invention by automatically switching to less visible red illumination light when the patient should start falling asleep or has been fallen asleep. The switch could be made manually (e.g. by hospital staff), but is preferably made automatically, using means (such as actigraphy means) and/or an algorithm that determines the state of awakens of the patient using actigraphy patterns as input. For instance, the image data obtained by the camera 18 may be used to determine if the subject 14 is awake or sleeping, e.g. based on a recognition of the breathing pattern, of movements and/or of the state of the eyes (closed or open, eye lid movements). In other embodiments, separate means for movement or breathing detection may be used.

Two scenarios, awake and sleep, each with their preferred choices of wavelengths, are distinguished in an embodiment. In the awake scenario red light at 660 nm and IR light at 800 nm are preferably used, providing a good SpO2 contrast and good motion robustness, but being visible. In the sleep scenario red light at 750 nm and IR light at 800 nm are preferably used, providing a smaller SpO2 contrast and smaller motion robustness, but being invisible.

The switch between the two red wavelengths for the two scenarios could also be made gradually in smaller steps rather than between just two wavelengths. Even a continuous adjustment of wavelengths, e.g. depending on the patient motion conditions, could be made.

IR light of 800 nm is not necessarily preferred. In fact, a larger contrast is obtained when the IR is chosen at wavelengths larger than 800 nm. However, for the sake of visibility this is irrelevant since this wavelength is barely visible anyway.

Hence, according to this embodiment a combination of IR and red wavelengths for SpO2 that is less obtrusive when the situation permits it is proposed. An algorithm or a user, e.g. hospital, staff may decide when to switch between the states. Decisions to switch between the states could be based simply on the time (e.g. time to go to sleep or to wake up), the signal to noise ratio of the PPG signals or from motion data (e.g. actigraphy, using the camera or by other means) that indicates when the patient is getting drowsy. In the latter case, a gradual switch (instead of abrupt) from well visible red to less visible red could be beneficial to the patient and his/her attempt to fall asleep.

In summary, it is an idea of the proposed system and method are as unobtrusive and comfortable for the patient as possible, but are at the same time always offering the measurement performance that is required because of medical reasons. The system will only become a good system in the eyes of our customers if it offers a good balance between comfort and performance/safety. So the “logic” (i.e. the control unit) that decides when to switch to more obtrusive setting to provide a better measurement performance is configured accordingly.

In one embodiment the system is generally running in “unobtrusive mode” (also called “comfort mode”) by default. This could for example mean, that “invisible” wavelengths from the NIR range are used for illumination, only limited illumination strength is used, or only spot illumination on small skin areas is used, etc. Under one or more of the following conditions the system changes the settings to “best performance mode” (whereby it should be remarked that an actual implementation may comprise more than two discrete operating modes):
SpO2 starts to drop (in unobtrusive mode it is not known for sure how much the drop is, but a trend should be visible);
SNR is getting too bad to deliver measurement results at all (e.g. low pulsatility or ambient light interferences, other extrinsic noise);
One or more vital signs (e.g. heart rate, respiration) are getting out of a safe range;
Any other available indicator that the patient is starting to deteriorate (e.g. the patient makes unusual noise, shows unusual actigraphy patterns, etc.).

In general, the present invention seeks to find an optimum tradeoff or compromise between maximum safety/measurement performance and minimum obtrusiveness. Conventional systems, in contrast, generally try to provide optimum measurement performance. A preferred control rule may, however, be that in case of doubt the safety mode is used, i.e. if the system suspects that the patient is not doing well for any reason the “obtrusiveness” has very limited priority.

Various input parameters may be used for the control according to the present invention. In particular, one or more of the following input parameters may be used:
patient data: e.g. demographic data, information from the patient history, health records, current and historic physiologic measurement data and trends, laboratory data and trends, age, gender, size, weight, health status, prior measurements of vital signs, health data and other individual risk factors specific for a subject;
feedback of current measurement data of a parameter: e.g. decreasing SpO2 causes an automatic switching to best performance mode in order to confirm measurement data and/or increase their accuracy;
feedback of other physiological measurement data; e.g. the system simultaneously measures pulse rate and SpO2; if the pulse rate leaves a predefined range, not only the measurement of pulse rate, but also of SpO2 is switched to best performance mode, since it is assumed that the patients state is worsening and it is desired to obtain more reliable and accurate information about the patient's state;
physical state information of the subject: e.g. information if the patient is moving, not moving, sleeping, awake;
signal quality: e.g. if no vital sign can be derived from the measurement data because the SNR is too bad, the system has to be controlled to switch (e.g. gradually) to best performance mode until the SNR is improving and/or the measurement of data is possible.

Various output parameters may be used by the control according to the present invention. Preferably, one or more parameters of the illumination source 22, the detection unit 18 and/or the analysis unit 34 may be controlled such that the accuracy and reliability of one or more derived vital signs are increased in case of a decreasing health state of the subject and/or a decreasing quality of the input signals or the derived vital sign, and/or such that the unobtrusiveness of the illumination and detection of radiation is increased in case of a stable or increasing health state of the subject and/or a stable or increasing quality of the input signals or the derived vital sign.

In particular, one or more of the following output parameters may be used:
intensity, wavelength, direction and/or illumination angle (focus and/or size of illuminated region of interest) of light emitted by illumination source 22;
acquisition rate, exposure time, focus, zoom or active sensing area of the detection unit 18;
switching between continuous and non-continuous data acquisition;
mode of the analysis unit 34 which vital signs to derive from the PPG signals;
change of data acquisition method (e.g. perform data acquisition additionally or alternatively with other data acquisition means; e.g. inform the nurse that the current measurement does not provide the necessary performance, so that a conventional measurement could be initiated by the nurse).

By way of example, the present invention can be applied in the field of health care, e.g. unobtrusive remote patient monitoring, general surveillances, security monitoring and so-called lifestyle environments, such as fitness equipment, or the like. Applications may include monitoring of oxygen saturation (pulse oximetry), heart rate, blood pressure, cardiac output, changes of blood perfusion, assessment of autonomic functions, and detection of peripheral vascular diseases. The present invention can particularly be used for rapid and reliable pulse detection of a critical patient, for instance during automated CPR (cardiopulmonary resuscitation). The system can be used for monitoring of vital signs of neonates as well. In general, the present invention allows both spot-check and continuous monitoring.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. Device for determining vital signs of a subject comprising:
an interface for receiving input signals from a detection unit that is configured for contactless detection of radiation reflected from a subject in response to illumination by an illumination source,
a processing unit for deriving photoplethysmography, PPG, signals from the received input signals,
an analysis unit for deriving a desired vital sign from the PPG signals,
a control unit for switching the illumination source between at least a safety mode and a comfort mode and for controlling one or more parameters of the illumination source according to the respective mode, in dependence on the health state of the subject,
wherein said control unit is configured to switch the illumination source into the safety mode, in which one or more derived vital signs can be derived with increased accuracy and reliability compared to the comfort mode, in case of a decreasing health state of the subject and to switch the illumination source into the comfort mode, in which the illumination has increased unobtrusiveness compared to the safety mode, in case of a stable or increasing health state of the subject.

2. (canceled)

3. Device as claimed in claim 1,
wherein said control unit is configured to control one or more of the intensity, wavelength, direction and/or illumination angle of light emitted by illumination source.

4. Device as claimed in claim 1,
wherein said control unit is configured to control acquisition rate, exposure time, focus, zoom or active sensing area of the detection unit.

5. Device as claimed in claim 1,
wherein said control unit is configured to control the analysis unit which vital signs to derive from the PPG signals.

6. Device as claimed in claim 1,
wherein said interface is configured to receive a sequence of image frames as input signal acquired by an imaging unit, in particular a camera, and wherein said analysis unit is configured to obtain physical state information of the subject from said sequence of image frames.

7. Device as claimed in claim 1,
wherein said analysis unit is configured to determine if the subject is in a physical state of moving or not moving, in particular in a physical state of sleeping or awake.

8. Device as claimed in claim 1,
wherein said control unit is configured to additionally use personal data of the subject, in particular age, gender, size, weight, health status, prior measurements of vital signs, health data, for controlling one or more parameters of the illumination source, the detection unit and/or the analysis unit.

9. System for determining vital signs of a subject comprising:
an illumination source for illuminating the subject,
a detection unit for contactless detection of radiation reflected from a subject in response to said illumination to obtain input signals, and
a device as claimed in claim 1 for determining vital signs of the subject.

10. Method for determining vital signs of a subject comprising:
receiving input signals from a detection unit that is configured for contactless detection of radiation reflected from a subject in response to illumination by an illumination source,
deriving photoplethysmography, PPG, signals from the received input signals,
deriving a desired vital sign from the PPG signals,
switching the illumination source between at least a safety mode and a comfort mode and controlling one or more parameters of the illumination source according to the respective mode in dependence on the health state of the subject, wherein the illumination source is switched into the safety mode, in which or more derived vital signs can be derived with increased accuracy and reliability compared to the comfort mode, in case of a decreasing health state of the subject and wherein the illumination source is switched into the comfort mode, in which the illumination increased unobtrusiveness compared to the safety mode, in case of a stable or increasing health state of the subject.

11. Computer program comprising program code means for causing a computer to carry out the steps of the method as claimed in claim 10 when said computer program is carried out on the computer.
